(19) United States
(12) Patent Application Publication
(10) Pub. No.: US 2013/0337095 A1
(43) Pub. Date: Dec. 19, 2013

(54) ANTIMICROBIAL COMPOSITION AND ITS METHOD OF USE

(71) Applicant: Waikatolink Limited, Ruakura Road (NZ)

(72) Inventor: Nichola Robyn Harcourt, Hamilton (NZ)

(73) Assignee: Waikatolink Limited, Ruakura Road (NZ)

(21) Appl. No.: 13/920,485

(22) Filed: Jun. 18, 2013

Related U.S. Application Data
(60) Provisional application No. 61/661,026, filed on Jun. 18, 2012.

(51) Int. Cl. A61K 36/61; A61K 36/52; A61K 36/15

(52) U.S. Cl. CPC A61K 36/61; A61K 36/15; A61K 36/52
USPC 424/770; 424/725; 424/771

(57) ABSTRACT

An antimicrobial composition is provided, including melaleuca oil, or an isolated fraction or an active agent or equivalent thereof, manuka oil, or an isolated fraction or an active agent or equivalent thereof, and at least one galloyl moiety.

### RELATED APPLICATION

This application claims 35 USC 119(e) priority from U.S. 61/661,026, filed Jun. 18, 2012.

### TECHNICAL FIELD

The present invention relates to antimicrobial compositions and methods of use.

### BACKGROUND

There are many clear advantages of currently available antibiotics (targeting bacteria) and broader antimicrobials agents or compositions (which can target not only bacteria but also fungi and viruses). Yet, as will be outlined further below, there are a number of problems associated with conventional antibiotics and antimicrobials, which prompts the need to develop effective alternatives to treat microbial infections beyond those currently available, particularly those for treating bacteria.

Firstly, bacteria are able to build resistance to antibiotics over time. The infection caused by the same bacteria is often harder to treat in the same or other individuals, rendering many currently available antibiotic treatments to be of lesser use or no use in future. Therefore, doctors and veterinarians are cautious about prescribing antibiotics unless necessary to avoid a build up of resistance by the microbes. This is also true for fungi which can develop resistance to antifungal drugs.

Secondly, antibiotics must also be applied according to the specific bacterial genera present, as the antimicrobial action may have narrow action on a specific structural feature of the bacterium. Yet, in many cases such as a microbial skin infection there may be more than one bacteria or even fungi that colonise the site. Viruses can also cause skin disorders. This means that antibiotics can be quite restrictive in their effective use.

Thirdly, antibiotics can lead to adverse side effects when used externally or internally. Some adverse side effects include diarrhea, nausea and vomiting. Itching and burning can occur with external antibiotic application.

The general public can also have a mistrust of antibiotics in part due to systemic administration, which is often not necessarily required to overcome the infection.

Therefore, significant research and development has been undertaken in the identification, testing and commercial application of naturally derived antimicrobial agents as an alternative to treat bacterial and microbial infections.

Generally speaking, naturally occurring antimicrobials have a much greater level of public acceptance than antibiotics (which target bacteria specifically) owing to the fact they are perceived as being less likely to harm an individual.

There is also a growing perception that natural antimicrobial agents are less likely to cause side-effects than synthetic drugs.

Furthermore, natural products may have significant advantages over synthetic agents because they potentially offer a multipronged approach to microbial control which may include a number of different actions such as direct cytocidal effects, interruption of the cell wall, elimination of plasmids conferring resistance and interference with efflux pump proteins.

There has been a substantial growth in popularity towards naturally derived antimicrobials also due to the recent push to become more sustainable. In other words, there is a publicly-led campaign to utilise naturally occurring material in our environment as opposed to developing new active agents in the laboratory. The main desire of consumers is to become more environmentally aware and make more efficient use of resources available.

Also, identification of naturally occurring antimicrobial agents can help to lower research and development costs. In many cases, it can be more cost and time-effective to identify and improve a compound or extract with desired characteristics and traits than trying to develop one through rational drug design.

Despite these advances, many naturally-derived antimicrobial compositions (and even some antibiotics) still have low potency, low persistency and/or still have a narrow spectrum of conditions/microbes against which they can be therapeutically used. Some examples of naturally derived antimicrobial compositions are provided below.

U.S. Pat. No. 6,514,539 discloses an antimicrobial composition that includes the use of manuka oil and melaleuca oil. The inventive concept was identifying an apparent synergistic effect between these two components. However, it is considered by the present inventors that no apparent synergy is present between these two oils. Instead melaleuca oil is known to target gram-negative bacteria, while manuka oil targets gram-positive bacteria. Hence, by combining them, the result is the two oils can target a wider range of bacterium. This is not a synergism, merely the cumulative result of the effect of the two oils.

US 2010/0324151 discloses compositions for treating infections in the eyelids, the ocular surface, skin and the ear caused by microbes such as Staphylococcus and Pseudomonas. The disclosed compositions include melaleuca oil. The description of the invention is primarily focused on eye treatment, which requires very low concentrations of antimicrobial agent to avoid toxicity issues due to the sensitivity of the local environment. Due to these low concentrations needed to avoid toxicity, it may present problems with providing the required potency and persistency.

US 2012/0009284 discloses a sprayable antimicrobial composition with the main active agents identified as melaleuca oil and peppermint oil. These oils are included to make the treatment more broad spectrum and no synergy was reported.

US 2007/0207112 discloses an anti-acne cream with anti-microbial peptides, optionally together with rice-bran extract, boswellia extract, manuka honey and melaleuca oil. The combination is a targeted treatment for a single condition, namely acne, with two parts to the treatment including antimicrobial action against P. acnes specifically and anti-inflammatory activity to resolve the inflammation that accompanies the acne bacterium in this condition. There are no claims for action against broader microbes, and there are no synergistic effects identified.

One particular example of a need to develop natural alternatives to antibiotics is for the treatment or prevention of ear infections in animals, including otitis externa. This is often difficult to treat with conventional antibiotics when the infection is caused by Pseudomonas aeruginosa.

Pseudomonas aeruginosa is found in soil, water, and on skin. It can live in a multitude of conditions and cause diseases in humans and many other animals. Importantly, the bacterium is multi-drug resistant and is therefore a difficult organism to kill or to inhibit its growth.

It is desirable to develop compositions able to effectively target both gram-positive and gram-negative bacteria with enhanced persistency and potency (and at minimum concentrations that avoid toxicity).

Naturally derived antimicrobial compositions have applications in other industries, including but not limited to the cosmetic, food and beverage, sanitizer and cleaning industries. There is a need to develop new products that have improved antimicrobial activity. This can help to reduce volumes of active components required, lengthen the shelf life of products, improve hygiene levels in hospitals, in the home environment and so forth.

It is an object of the present invention to address the foregoing problems or to at least provide the public with a useful alternative choice.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

Throughout this specification, the word “comprise”, or variations thereof such as “comprises” or “comprising”, will be understood to imply the inclusion of a stated element, integer or step, or group of elements integers or steps, but not the exclusion of any other element, integer or step, or group of elements, integers or steps.

Further aspects and advantages of the present invention will become apparent from the ensuing description which is given by way of example only.

### SUMMARY

According to one aspect of the present invention there is provided an antimicrobial composition including at least:
a) melaleuca oil, or an isolated fraction or an active agent or equivalent thereof;
b) manuka oil, or an isolated fraction or an active agent or equivalent thereof; and
c) at least one galloyl moiety.

According to a further aspect of the present invention there is provided a method of manufacturing an antimicrobial composition characterised by the steps of combining at least:
a) melaleuca oil, or an isolated fraction or an active agent thereof;
b) manuka oil, or an isolated fraction or an active agent thereof; and
c) at least one galloyl moiety.

According to a further aspect of the present invention there is provided a use in the manufacture of any one of the antimicrobial compositions as described above for the prevention or treatment of a microbial infection in an animal in need thereof.

According to a further aspect of the present invention there is provided a method of preventing or treating a microbial infection in an animal;

characterised by the step of administering to the animal a therapeutically effective amount of any one of the antimicrobial compositions as described above.

### Outline of Advantages of the Present Composition

It is known that each of the components used in the present invention has antimicrobial activity.

However, for the first time, it has been identified by the inventors that there is a surprising synergistic antimicrobial effect when the compositions as described above were tested in vitro.

When compared to the effect of each component individually, the compositions have an impressive effect in terms of potency and persistency. These preliminary results are clearly exemplified in the Best Mode section.

The inventors identified the most likely candidates for the synergistic effect observed are the ketone moieties and the monoterpene moieties, which are present in manuka oil and melaleuca oil respectively, and galloyl moieties in the black walnut hull.

These happen to be a convenient and rich source of the proposed active agents; however, the present invention should not be limited to such. The inventors acknowledge that potentially any source of these active agents (both synthetically or naturally derived) may be used in the present invention. Further studies are being performed to support this.

The inventors noted a clear synergistic antimicrobial effect in a composition containing melaleuca oil, manuka oil and a galloyl moiety (with preferred sources from walnut hull—preferably black walnut hull, and/or pine bark extract). However, someone skilled in the art would also expect that, after reading the present specification and reviewing the preliminary results, the synergy would be present just as a combination of a galloyl moiety and one ketone moiety or one monoterpene moiety (or sources thereof such as black walnut hull together with either manuka oil or melaleuca oil).

This is because the most likely active agents in manuka oil and melaleuca oil are ketones and monoterpenes, and also that no synergy was seen between these two oils when in combination without walnut hull.

Compared to compositions made up of manuka oil and melaleuca oil, the inventors recorded an approximately 16-fold increase in the potency of the present composition in their preliminary trials. In other words, the same level of antimicrobial effect was observed when using a composition having a 16 fold dilution of the total active agents (% v/v). This opens up many potential opportunities.

For instance, it allows the use of the composition for treating conditions where high concentrations of active agents leads to toxicity, such as discussed in relation to eye conditions or other sensitive external regions on the body.

It also may allow smaller volumes to be administered where high concentrations are not an overriding concern.

The greater persistency also illustrates a potential advantage in requiring a less stringent dosage regime with fewer treatments. Other advantages are also envisaged.

The composition of the present invention also shows remarkable effectiveness even compared to antibiotics such as gentamicin and ciprofloxacin. This is a significant development as it:
a) relies entirely on components that are naturally derived,
b) uses components that are already therapeutically acceptable, used for animal and human use,
c) avoids several disadvantages of antibiotics,
d) uses a multi-pronged mode of action that reduces the likelihood of developing resistance in comparison to antibiotics,
e) targets a broader range of organisms than antibiotics.

As outlined further, the inventors acknowledge that the composition may be used for treating or preventing substantially any external microbial infection in any animal.

Furthermore, the composition may be used for non-therapeutic purposes. The unique combination of components may be used for the likes of food preservation, make-up, household disinfectants, hand sanitizers, soaps, deodorant, body washes, mouth washes, dental care, shampoos/hair products, face packs, or substantially any other use which commonly utilise or include antimicrobial components. Some examples of potential applications of the present invention are referred to further in this specification.

Further aspects of the present invention and its advantages will become clear with the ensuing description.

### DEFINITIONS

Throughout this specification the term antimicrobial composition should be taken as meaning any combination of substances which kills or inhibits the growth of a microorganism such as a virus, bacteria, or fungus. The composition may be “microbiocidal”, meaning that it kills the microbes, or “microbiostatic”, meaning that it prevents the growth of microbes, or a combination thereof.

In particular, the present invention has been shown to be very effective against Pseudomonas aeruginosa. P. aeruginosa is particularly hard to treat because it is already naturally resistant to a large variety of antibiotics. It has an outer membrane which contains Protein F (OprF), a porin, providing its outer membrane with an exclusion limit of 500 Da, thereby lowering the permeability of the outer membrane, and decreasing the intake of harmful substances into the cell.

P. aeruginosa has a number of molecular pumps that remove any drug that enters the cytoplasm. P aeruginosa is multidrug resistant via several additional mechanisms including: the acquisition of genes that produce extended spectrum beta lactamases (eSbLs), enzymes that break down antibiotics and prevent them from working, production of enzymes that modify aminoglycoside antibiotics, or break down cephalosporin antibiotics (cephalosporinases) or carbapenem antibiotics (carbapenemases) and changes to proteins that help make bacterial genetic material can confer resistance to quinolones or other compounds.

Pseudomonas may form biofilms, which enable bacteria to change the biochemical landscape in a way that it enhances the pathogenicity of the bacteria, increasing the complexity and number of bacteria that form the biofilm. The ability of P. aeruginosa to use numerous mechanisms of antibacterial resistance and its ability to acquire additional determinants further compound the complexity of managing this organism.

It is a well-known practice to test antimicrobial compositions against Pseudomonas because if they are found to be effective against same, then it is a reasonable extrapolation that the composition will be effective against other gram negative bacteria.

As the present invention has effective antimicrobial activity it is expected that it will also have potent activity against other bacteria which are difficult to kill. Further, that the present invention has potent activity against Staphylococcus. aureus I it is reasonably expected by those skilled in the art to be effective against other gram positive bacteria for the following reasons.

All bacteria of pathogenic significance share a number of common structures that convey protection from toxic chemicals, and these include efflux pumps and cell walls comprised of peptidoglycan. Thus possessing both peptidoglycan cell walls and efflux pumps are highly conserved features of bacteria in general. Where control agents are known to interfere with the integrity of the peptidoglycan cell wall, it follows that the exposure of all bacteria to these agents will weaken the bacterial cell defense and enable chemical compounds that once would have been excluded to now penetrate the bacterial cell.

Once toxic agents have entered the bacterial cytoplasm it is then possible for the compounds to interfere with the function of the efflux pumps, thus enabling toxins to accumulate and exert cytocidal effects.

It is more challenging for chemicals to reach the cytoplasm of gram-negative bacteria because they have an additional barrier in the form of an outer membrane containing lipopolysaccharides. It therefore follows that a chemical agent that exhibits potent antimicrobial activity against a gram-negative bacterium could be expected to be at least as effective when used to treat a gram-positive bacterium. This could reasonable be expected because a gram-positive bacterium is structurally less complex than a gram-negative bacterium due to the absence of the lipopolysaccharide membrane.

Throughout this specification the term “ketone moiety” should be taken as meaning any compound which includes the general structure RC(═O)R′, where R and R′ may be a range of carbon-containing substituents, as represented schematically below.

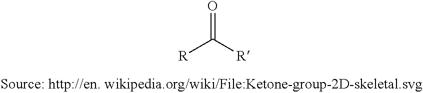

The inventors envision the ketone moieties which contribute significantly to the synergistic effect in combination with the at least one galloyl moiety are the di- and/or tri-ketones. However, it should be understood that other ketones would be expected to also provide a synergy as identified by the present invention. This definition should be understood to encompass both synthetically derived ketones, as well as naturally derived ketones, either fully, partially, or non-isolated from an original source, and including any derivative or modification thereof. Further examples will be provided below.

Throughout this specification the term “monoterpene” should be taken to mean any compound from a class of terpenes that consist of two isoprene units and have the molecular formula C₁₀H₁₆. Monoterpenes may be linear (acyclic) or contain rings. Biochemical modifications such as oxidation or rearrangement produce the related monoterpenoids.

Throughout this specification the term “galloyl moiety” should be taken as meaning any compound which includes gallic acid as part of the compound. A gallic acid, also known as 3,4,5-trihdroxybenzoic acid, is a trihydroxybenzoic acid which is a type of phenolic acid (a type of organic acid).

Some plants which are in general found to have high galloyl moieties include: Acer sp. (maple), Betula sp. (birch), Salix caprea (willow), Pinus sp. (Pine), Sorghum sp., Boswellia dalzielii, Drosera, Rhodiola rosea, Triphala, Toona sinensis.

However, a number of common foods can also include high galloyl moieties such as Areca nut, Bearberry (Arctostaphylos sp), Bergenia sp, Blackberry, gallnuts, Hot chocolate, Juglans regia (Common walnut), Mango in peels and leaves, Phyllanthus emblica (Indian gooseberry) in fruits, Raspberry, Syzygium aromaticum (clove)^{[16]}, Vinegar^{[17]}, wine, Witch hazel (Hamamelis virginiana), sumac and White tea.

The chemical formula of gallic acid is C₆H₂(OH)₃COOH, as represented schematically below. Gallic acid is found both free and as part of tannins, both of which are encompassed within the scope of the term galloyl moiety as used herein.

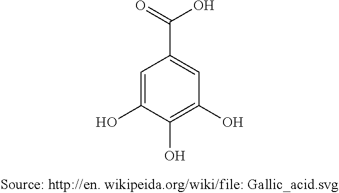 [SMILES: O=C(O)c1cc(O)c(O)c(O)c1]

Salts and esters of gallic acid are termed “gallates” and are also encompassed within the scope of the term galloyl moiety. The process of esterification with gallic acid is termed galloylation. Therefore, this definition should be understood to encompass both synthetically derived galloyl moieties, as well as naturally derived galloyl moieties, either fully, partially, or non-isolated from an original source.

Throughout this specification the term “ . . . an oil from a plant of the Myrtaceae species” should be taken as meaning any oil which is extracted from any plant belonging to the Myrtaceae (or Myrtle) family.

Examples of such plants include those of the Leptospermum genus which includes over 80 species of plants, such as Leptospermum scoparium (more commonly known as manuka or tea tree) or Leptospermum morrisonii.

Another example of plants within the Myrtaceae species include melaleuca alternifolia, melaleuca dissitiflora and melaleuca linariifolia. These plants are examples of those which are known to produce melaleuca oil (also known as tea tree oil). The most common household names for melaleuca alternifolia are Narrow-leaved Paperbark, Narrow-leaved Tea-tree, Narrow-leaved Ti-tree, or Snow-in-summer and are generally found in Australia (http://en.wikipedia.org/wiki/melaleuca_alternifolia).

Other plants belonging to the Myrtaceae include clove, guava, feijoa, and eucalyptus. This definition should include (but not necessarily be limited to) any plants in this Myrtaceae species.

Other antimicrobial agents known to be within oils such as melaleuca oil and manuka oil are outlined further in this specification.

Throughout this specification the term “walnut hull” should be taken as meaning the hull (meaning the outer soft fruit encasing the shell and nut) of a walnut. It should not be confused with walnut oil, which is a different component to the hull of a walnut.

The genus Juglans contains more than 25 species. The species Juglans nigra (black or American walnut) and Juglans regia (English walnut) are preferred. There is much literature for the hull extracts of these two species having broad antimicrobial activity, which are known to include galloyl moieties which is considered by the inventors to play a role in the synergistic antimicrobial effect observed. However, other walnut species common to the genus Juglans may be used as the source of active agents contributing to the synergistic antimicrobial effect observed.

Black walnut is so closely related to the English walnut that they are virtually indistinguishable medicinally.

It should be noted that the present invention has been found to be effective using Pinus radiata bark extract.

### Preferred Embodiments of the Corn Position

The inventors' preliminary study utilised manuka oil and melaleuca oil combined with walnut hull. This is because, as discussed previously, the antimicrobial profiles in manuka oil and melaleuca oil are known to be different and better suited for treating gram-positive and gram-negative bacteria, respectively. By using two or more oils from plants of the Myrtaceae family, it may help to widen the spectrum of microbes which may be effectively treated with one composition. Also, because a synergy is observed between galloyl moieties derived from walnut hull and a Myrtaceae plant oil, this synergy would be expected to be extended across a number of bacterial species if more than one Myrtaceae plant oil is present in the composition.

Preferably, the at least one galloyl moiety is from a black walnut hull. An alternative is that the galloyl moiety may be synthetically derived or extracted from other plants/bark/shells/husk/plant waste as discussed previously.

As noted previously, an alternative source which works particularly well is galloyl moieties from pine bark extracts.

In a particularly preferred embodiment, the composition includes melaleuca oil, manuka oil and walnut hull.

Without wishing to be bound by theory, it is possible that one or more components from each oil/hull is contributing significantly to the synergistic effect. By avoiding any fractionation/purification steps of each component, it is possible that this may heighten the synergistic effect observed.

Other active agents which may be contributing to the underlying synergistic effect are provided in the table below.

| Source | Proposed active(s) |
|---|---|
| melaleuca oil | Monoterpenes (including alpha-pinene, beta-pinene, |
| (tea tree oil) | alpha-terpineol, terpinen-4-ol and linalool) and |
| | sesquiterpenes |
| manuka oil | Ketones (e.g. triketones including leptospermone, |
| | Isoleptospermone and flavesone and/or diketones), |
| | sesquiterpenes and linalool. |
| walnut hull | Tannins such as galloyl, juglone (5-hydroxy- |
| | alphanapthaquinone), 1,4-naphthoquinone, 2-methyl- |
| | 1,4-naphthoquinone, plumbagin (5-hydroxy-2-methyl- |
| | 1,4-naphthoquinone), iodine, phenolic compounds such |
| | as naphtoquinones, flavonoids, chlorogenic acid, |
| | caffeic acid, ferulic acid, sinapic acid, gallic acid, ellagic |
| | acid, protocatechuic acid, syringic acid, vanillic acid, |
| | catechin, epicatechin, and myricetin |

This is supported by research which shows in some cases the activity and perhaps synergistic effect may be lost when fractions which contain the active agents are purified (Cos et al., Antiinfective potential of natural products: How to develop a stronger in vitro proof of concept. J. Ethnopharmacol. 2006. 106: 290-302).

A further added benefit of this embodiment is it may help to avoid processing time, and therefore costs.

Alternatively, the composition includes a fraction or isolated portion of the melaleuca oil, manuka oil and/or walnut hull.

The fraction or isolated portion may contain at least one active agent, but more likely a number of active agents, possibly together with other components of the oils or hull.

A benefit of isolating a fraction may be that it can be used to provide a high potency fraction to increase the strength of the composition. This may be because one or more active agents may be isolated in a smaller fraction. This may be useful for some applications which require higher concentrations of one or more of the components.

The fraction or isolated portion may be derived through a range of common scientific techniques such as size exclusion chromatography, affinity chromatography, supercritical CO₂ extraction, dimethyl ether extraction and so forth. It should be appreciated that the amount of each component (and ratio thereof) may vary depending on the target organism and/or condition to be treated.

In a preferred embodiment of the present invention, the antimicrobial composition (excluding excipients) has the percentage of manuka oil in the order of 24% to 34% v/v.

In a preferred embodiment of the present invention, the antimicrobial composition (excluding excipients) has the percentage of melaleuca oil in the order of 62% to 72% v/v.

The respective ratios of the two oils (namely manuka and melaleuca) may change dependent on the species being targeted. For example, gram negative and gram positive bacteria require different optimal ratios.

In a preferred embodiment of the present invention, the antimicrobial composition (excluding excipients) has the percentage of walnut oil in the order of 2 to 6% v/v.

It should be appreciated that as discussed later that the composition may include a number of additional excipients. However, by providing percentages for the other excipients given gives an appreciation of the ratio of the compounds to each other that gives the synergistic effect.

In one embodiment the inventors found that for activity against Pseudomonas, the composition (without excipients) preferably included approximately 4% v/v walnut hull, approximately 29% v/v manuka oil and approximately 67% v/v melaleuca oil.

These concentrations reflect U.S. Pat. No. 6,514,539 which discusses the preferred ratio of manuka oil to melaleuca oil for killing broad spectrum microbes was 30:70%.

The preferred 4% content of walnut hull extract was chosen based on calculations by the inventors, so that the final concentration of walnut hull extract in the product was 200 mg/ml dried plant extract (DPE).

However, it should be appreciated that a wide range of concentrations of each component (or fraction/active thereof) may be utilised in the present invention to achieve a synergistic antimicrobial effect, or such that is particularly adapted to treating a certain microbe or condition resulting from same. Such tests could easily be performed by a person skilled in the art.

Preferably, the composition is formulated as a topical composition. The inventors acknowledge that any common excipients used in topical compositions may be applied to the present invention without departing from the scope thereof.

More preferably, the topical composition is in the form of an oil, cream, liquid, paste, solution, spray, gel, wound care dressing, drops or ointment, etc.

Most preferably, the topical composition is in the form of a cream, spray, cleanser or drops.

In a further embodiment, the composition includes at least one excipient.

It is envisioned by the inventors that substantially any excipients or combination thereof may be used as commonly applied in the pharmaceutical industry for internal or external medicaments.

Such excipients may include penetration enhancers, carriers, surfactants, stabilisers, enhancers, thickeners, solvents, antioxidants, and so forth.

If the composition includes excipient(s), the final concentration of the melaleuca oil, manuka oil and walnut hull may vary considerably, and of course would depend on the type and severity of the condition to be treated.

Also, it would be understood by someone in the industry that the composition may be adjusted to have a suitable density and/or viscosity for the treatment and condition presented. Viscosity modifiers or thickeners may be utilised to achieve these variables.

In the embodiment wherein active agents are used (instead of the whole oils/hull), the inventors consider the most likely concentration of each active agent is between 0.001 to 50% w/v. Most preferably, the concentration of each active agent is between 0.1 to 10% w/v.

In still a further embodiment, the composition includes at least one additional active agent.

For example, the additional active agents may include antimicrobial agents, analgesics, anti-inflammatory agents, anti-fungal agents, antibiotics, and so forth. For instance, alternative antimicrobials may be added to provide an ingestible format, the composition may be combined with antibiotics to reduce antibiotic dosage requirements, or the composition may be combined with anti-fungal agents to efficaciously target a broader range of microbes.

### Preferred Embodiments of the Method of Treatment and Use

Preferably, the method of treatment includes administering the composition topically to an animal. The inventors envision a particularly useful application of the composition is for treating skin disorders and infections in a human or animal. However, this should not be limiting as there are significant other applications and uses of the present invention.

Throughout this specification, the term topical treatment should be understood to mean non-systemic indications, for instance to a surface which does not require the active agents to cross the intestinal wall and enter the bloodstream. Obvious examples include the skin, mouth, eyes, ears, vagina, rectum and so forth. The term topical treatment should also be understood to include treatments to areas such as within the udder of an animal (for instance as mastitis treatment) or anywhere along the intestinal tract as long as the active agents do not cross the intestinal wall and enter the bloodstream.

Preferably, the method of treatment is for preventing or treating an infection caused by a gram positive bacteria or gram negative bacteria.

Preferably, the method of treatment is for preventing or treating an ear infection. Most preferably, the ear infection is otitis externa (otherwise known as external otitis or swimmer's ear).

Preferably, the composition is administered into the ear canal of an animal in need thereof.

The results provided in this specification clearly show a remarkable effect against Pseudomonas aeruginosa. In current studies currently underway by the inventors, a similar positive effect is being observed in other organisms (i.e. Staphylococcus aureus, and Candida albicans).

Despite the initial problem to be solved, it became apparent that the invention may be used for a range of external microbial infections in animals (including humans). By all accounts, the present invention should not be limited to the treatment of infections caused by Pseudomonas or specific conditions such as otitis externa.

One other preferred use of the compositions of the present invention include intra-mammary treatment and prophylaxis for mastitis. This may include application as an external prophy lactic teat seal, or as an internally applied treatment for the teat canal or udder.

It is within reasonable scientific prediction that the compositions of the present invention may also be useful in preventing or treating/preventing a range of indications and organisms as exemplified below:

| Organism | Key Indication |
|---|---|
| Pseudomonas aeruginosa | Infected wounds, Otitis media |
| Streptococcus pyogenes, | Pyoderma, muzzle folliculitis and |
| Staphylococcus | furunculosis, mastitis (E. coli) |
| intermedius, E. coli, | |
| Proteus | |
| Porphyromonas gulae, | Periodontal disease |
| P gingivalis | |
| Staphylococcus aureus, | Otitis externa, suppurative dermatitis, |
| Staphylococcus | abscesses, mastitis, pododermatitis, and |
| epidermidis | genital tract infections. This organism |
| | may cause pyogenic (abscessing) |
| | infections of the conjunctiva and adnexa |
| | of the eye, the skin and adnexa, or the |
| | genital tract. Mastitis (S. aureus) |
| Streptococcus uberis | Mastitis |
| Malassezia globosa, M. | Dandruff, atopic dermatitis, otitis |
| pachydermatis, M canis | externa |
| Trichophyton species | Pododermatis |
| Pasteurella multocida | Cat abscess |
| Candida albicans | Urinary infections and otitis externa. In |
| | an immune-compromised individual it may |
| | cause disseminated mycoses, local |
| | infection in perineum, nail folds, oral |
| | mucosa, cornea and the urinary tract. |
| | This organism may be transferred from |
| | owner to companion animal and vice versa. |
| Dermatophilus congolensis | Mudfever/Scratches (Staphylococcus spp. |
| | often involved) |
| Dermatophytes (including | Onychomycosis (fungal nail rot) |
| Epidermophyton, | |
| Microsporum, and | |
| Trichophyton species | |
| Tinea spp | Ringworm, athletes foot |
| Chlamydophila felis | Conjunctivitis, nasal discharge, respiratory |
| Streptococcus mutans | Tooth decay |

It should be noted that a trilogy at the ratio of 30:70:4 (manuka:melaleuca: walnut) works well against S. pyogenes, E. coli, S. uberis and S. agalactiae.

Additionally, similar to common uses of other antimicrobial compositions, the present invention may be used for a wide range of topical applications for the treatment or prevention of such ailments or conditions such as skin abrasions, abscesses, acne, bed sores, blisters, boils, burns, carbuncles, cold sores, cracked skin, dandruff, dermatitis, psoriasis, eczema, athletes foot, insect bites, lice, nail infections, pimples, ringworm, rhinitis, oily skin, sores, sunburn, tinea, tonsillitis, varicose ulcers and the like.

Preferably, the method of treatment has a dosage regime as a twice daily application to the affected area.

### Preferred Embodiments of the Method of Manufacture

In the broadest sense, the method of manufacture includes combining at least one oil from a plant of the Myrtaceae family and at least one galloyl compound.

In a particularly preferred embodiment where extracts are used, the method of manufacture includes combining at least a walnut hull (final concentration of approximately 4% v/v) melaleuca oil (final concentration of approximately 67% v/v), and manuka oil (final concentration of approximately 29% v/v).

Preferably, the method of manufacture includes adding a vehicle to the composition.

The vehicle may be selected from substantially any vehicle used in the pharmaceutical or cosmetics industries. However, the inventors foresee such vehicles as lanolin, aloe vera, hemp or flaxseed cream base may be particularly suitable.

However, the inventors foresee the three preferred components (manuka oil, melaleuca oil and black walnut hull) are unlikely to have stability issues when present together in a product.

### BRIEF DESCRIPTION OF DRAWINGS

Further aspects of the present invention will become apparent from the following description which is given by way of example only and with reference to the accompanying drawings in which:

FIG. 1 Provides a comparison of the % (v/v) test compound required of the novel plant extracts to obtain MIC90 against a reference strain of P. aeruginosa, and

FIG. 2 Provides a comparison of the % (v/v) test compound required of the novel plant extract formulation to obtain MIC90 against reference strains of various organisms.

### DETAILED DESCRIPTION

The antimicrobial composition of the present invention includes melaleuca oil, manuka oil and black walnut extract.

The composition is to be used as a topical cream or spray for the treatment of otitis externa in dogs which can be caused by Pseudomonas aeruginosa.

Of course, alternative uses for the present invention are envisaged, and should not be considered beyond the scope of this invention.

### Example 1

### Test Composition with No Excipients

| Component | Concentration (v/v) |
|---|---|
| manuka oil (Leptospermum scoparium) | 28.8% |
| melaleuca oil (melaleuca alternifolia) | 67.2% |
| black walnut hull (Juglans nigra) | 4% |
| TOTAL | 100% |

### Example 2

### Preliminary Study Against Pseudomonas aeruginosa

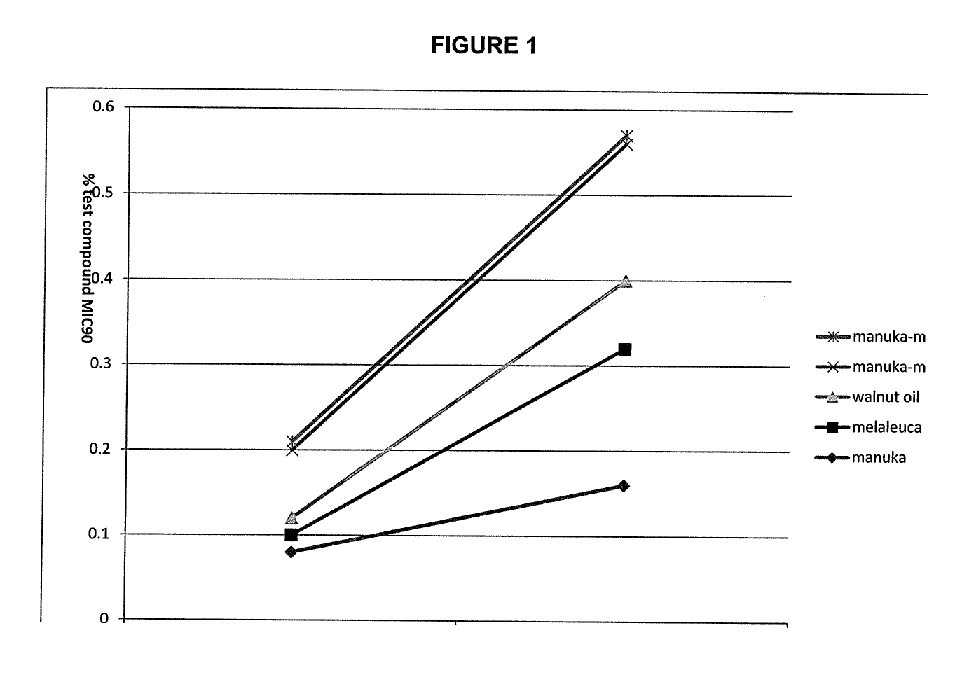

To exemplify the concept of the present invention, the in vitro activity of three novel plant extracts melaleuca oil, manuka oil, and black walnut hull extract, and blends of these extracts, were tested against an isolate of Pseudomonas aeruginosa from a reference library. MIC90 refers to the minimum inhibitory concentration required to inhibit the growth of 90% of the organisms. A graphical representation of the results is shown in FIG. 1. The methods and materials used for this preliminary study are outlined in Example 3.

TABLE 1
In-vitro anti P. aeruginosa activities of plant extracts
and antibiotics at MIC90 (% v/v stock solution)

| Sample | 18 h | 24 h |
|---|---|---|
| A. manuka oil | 0.08% | 0.16% |
| B. melaleuca oil | 0.02% | 0.16% |
| C. walnut hull extract | 0.02% | 0.08% |
| D. manuka-melaleuca | 0.08% | 0.16% |
| E. manuka-melaleuca-walnut | 0.01% | 0.01% |
| F. gentamicin | 0.098 μg/ml | 3.125 μg/ml |
| G. ciprofloxacin | 0.049 μg/ml | 0.098 μg/ml |

### Summary of Results:

In all cases, the MIC90 ranged from 0.01% to 0.16% (v/v); however there were significant variations in the MIC90 between each test composition used.

After 18 h of culture, MIC90 values were 0.01% for the blend of manuka-melaleuca-walnut, compared with 0.02% for melaleuca or walnut, and 0.08% for a blend of manuka-melaleuca or manuka alone. This indicates a higher potency of the composition having all three extracts.

After 24 h of culture, MIC90 values were still 0.01% for the blend of manuka-melaleuca-walnut, compared with 0.08% for walnut alone and 0.16% for manuka, melaleuca or the manuka-melaleuca blend.

The MIC90 remained the same at both incubation time-points for the novel blend of manuka-melaleuca-walnut, while the concentration of test solution required for the other extracts increased at least four fold after the cultures had been incubated for 24 h. This was a significant result as it illustrated the improved persistency as well as potency of the composition having all three extracts.

These studies indicate:
there was a synergistic effect obtained by creating a blend of manuka oil, melaleuca oil and walnut hull that was not obtained by any of the extracts tested alone or in pairs which were tested.
compounds obtained from the walnut hull extract exert antimicrobial activity beyond those compounds present in manuka-melaleuca.
the composition with the three extracts appear to be more potent, and also help to improved persistency of the composition compared to the other compositions tested.
a combination of isolated active agent(s) or at least certain fractions of the three extracts may still provide the synergistic effect exemplified here.
when compared to the manuka-melaleuca composition D, the addition of walnut hull in composition E (having all three extracts) appears to be a key to improving potency and persistency.
walnut hull together with either just manuka oil or melaleuca oil may also provide a synergistic effect with regards to potency and/or persistency (currently being tested).

### Example 3

### Materials and Methods

A third party was contracted to perform the independent testing described herein leading to the results outlined in this specification.

### 1. Description of Test Materials and Test Methods

The methodology used in this study is based on standard MIC assay methods referenced in numerous publications.
Each of the extracts was serially diluted to give 11 different concentrations. The diluted samples were then incubated with the Pseudomonas aeruginosa (ESR strain 981, NTC 10662, ATCC 25668, DSM 46358, NC/MB 13063). The MICs were determined as the lowest concentration of each extract that inhibits growth of the bacteria as determined by measurement of OD650 nm using a VersaMax 96 well plate reader.

### 2. Characterisation of the Test Systems:

Tryptic soy agar base (Bacto Difco Media Cat. No. 236940, Becton Dickinson and company).
Mueller II broth cation-adjusted (BBL media Cat. No. 212332, Becton Dickinson and company).

Pseudomonas aeruginosa (Gram negative).

3. Terminology Used with this Section
Substrate sterility controls (broth plus sample only)
Test substance free control (Mueller II broth plus inoculum) Microbial Purity control (final microbial inoculum streaked onto agar)

### 4. Sample Preparation and Assay Conditions:

a) The isolate of Pseudomonas aeruginosa was streaked from −70° C. cryostocks onto Tryptic Soy Agar (TSA) plates and incubated for 24-48 hrs at 37° C. until visible single colonies developed.
b) One colony was picked, using a sterile inoculating loop and used to inoculate sterile 10 ml universals of Mueller II Cation-adjusted broth. Inoculated broths were incubated for approx 18 hrs at 37° C.
c) Following overnight incubation the culture was diluted with fresh sterile Mueller II cation adjusted broth to an OD650 nm of approximately 0.1, equivalent to approx 105 CFU/ml (cell number and OD650 nm was determined and optimised prior to commencement of MIC testing). This is the seed broth which was used to inoculate the test wells in each plate. The seed culture was held at 4° C. until required for plating.
d) Stock solutions of each test extract were prepared so that their concentration is 64 μg/ml in the appropriate formulation in Mueller II cation adjusted broth. Once the extract was added to the broth it was mixed using a vortex mixer for approx 30 sec to ensure homogenous dispersion throughout the broth solution.
e) A 96 well microtitre plate was then set up as follows: To all wells, except the first in every row, 100 μl of sterile Mueller II cation adjusted broth was added. The plate was prepared up to 24 hrs in advance and held at −20° C. until ready for use.
f) If the plate was prepared in advance, it needed to be thawed at room temperature. To the appropriate first well in every row (see diagram below) 200 μl of the appropriate plant extract (containing 64 μg/ml of extract in Mueller II broth) was added.
g) Using a multichannel pipette, 100 μl of the plant extract was sampled from well 1 in each row and transferred to well 2, and mixed thoroughly by pipetting up and down 5 times. The pipette tips were discarded. Then, a fresh tip was added to the pipettor and 100 μl of solution was transferred from well 2 to well 3, mixed thoroughly by pipetting up and down 5 times and then the tips were discarded. This process was continued until the second to last well in each row. Discard 100 μl of solution from well 11 (leaving 100 μl as the final volume in this well). This process results in serial doubling dilutions that range from 64 μg/ml through to 0.06 μg/ml. The 12th and final well in each row will contain Mueller II broth only and will not be inoculated with seed culture. This well served as a sterility control and blank for each row.
h) Once the dilutions of the last test item were complete, 100 μl of the microbial seed culture was added to wells 1-11 of each row. The addition of the seed culture halved the extract concentrations in each well giving final well concentrations ranging from 32 μg/ml-0.03 μg/ml.
i) At the end of each microtitre plate the following test substance free controls were performed. 100 μl of Mueller II cation adjusted broth was added to wells. To each well 100 μl of Pseudomonas aeruginosa inoculum was added. This control serves show that the bacterial culture was alive and healthy and the final OD650 nm the culture would obtain without any test substance inhibition.
j) The plates were then placed on a plate shaker for 1 minute to ensure even mixing of the microbial seed cultures with the test solutions. The OD650 nm of each plate was then read using a Versamax microtitre plate reader. This was recorded as the zero time reading.
k) The plates were then incubated for 24 hrs at 37° C. at which point the OD650 nm of each well was read using a Versamax plate reader and recorded as the 24 hr reading.
l) The microtitre plate was then returned to the incubator for a further 24 hr and the OD650 nm was read and recorded again as the 48 hr reading.
m) Once the ODs of the plates had been read the wells containing the highest dilution of each sample (lowest concentration of test extract) without a detectable change in OD in comparison to the initial reading at time zero was noted.

### Experimental Design

Each of the three test items were assayed in duplicate against the bacterial isolate at 11 different concentrations. For each extract the first well in the plate (lowest concentration) that showed no change in OD650 nm after 24 and 48 hr incubation as compared to the zero reading were determined. These are reported as % of the extract stock solution. Appropriate controls were also run in triplicate.

### Example 4

### Preliminary Study Against a Broad Range of Microbes

To exemplify the concept of the present invention, the in vitro activity of three novel plant extracts melaleuca oil, manuka oil, and black walnut hull extract in a specific ratio described herein, were tested against an isolate of various microbes (methicillin-sensitive S. aureus, methicillin-resistant S. aureus, S. pyogenes, S. agalactiae, S. uberis, E. coli, C. albicans) from a reference library.

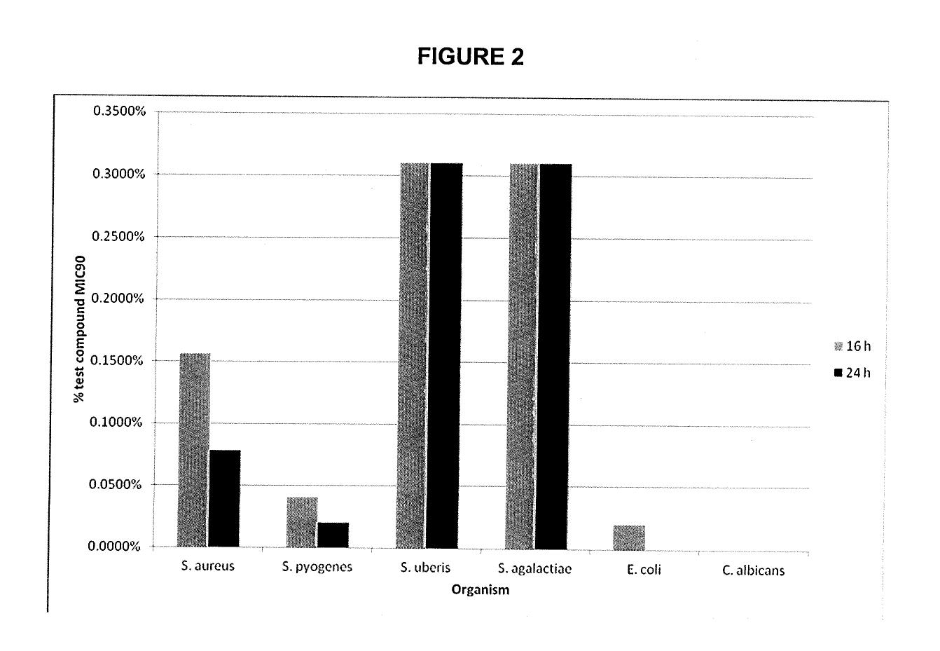

MIC90 refers to the minimum inhibitory concentration required to inhibit the growth of 90% of the organisms. A graphical representation of the results is shown in FIG. 2. The methods and materials used for this preliminary study are outlined in Example 5.

TABLE 2
In-vitro anti microbial activities of plant extracts
and antibiotics at MIC90 (% v/v stock solution)

| | Concentration | Concentration | | |
|---|---|---|---|---|
| | require to | required to | MIC antibiotic | MIC antibiotic |
| | achieve MIC | achieve MIC | reference | reference |
| Organism | 16 h | 24 h | 16 h | 24 h |
| S. aureus | 0.1560% | 0.0780% | Streptomycin at | Streptomycin at |
| | | | 0.003 ug/ml | 7.66 × 10−⁴ ug/ml |
| S. aureus | 0.625% | 1.25% | Doxycycline at | Enfrofloxacin at |
| (methicillin | | | 0.05 μg/ml | 0.05 μg/ml |
| resistant) | | | | |
| S. pyogenes | 0.0400% | 0.0200% | Penicillin at | Penicillin at |
| | | | 0.049 ug/ml | 0.049 ug/ml |
| S. uberis | 0.31% | 0.31% | Penicillin at | Penicillin at |
| | | | 0.049 ug/ml | 0.049 ug/ml |
| S. agalactiae | 0.31% | 0.31% | Cephalexin at | Cephalexin at |
| | | | 0.098 ug/ml | 0.098 ug/ml |
| E. coli | 0.0200% | <0.005% | Ciprofloxacin at | Ciprofloxacin at |
| | | | 0.049 ug/ml | 0.049 ug/ml |
| C. albicans | <0.005% | <0.005% | Amphotericin B at | Amphotericin B at |
| | | | 7.66 × 10−4 ug/ml | 3.83 × 10−4 ug/ml |

### Summary of Results:

In all cases, there was antimicrobial activity against the full range of microbes tested (S. aureus (methicillin-sensitive and methicillin-resistant), S. pyogenes, S. uberis, S. agalactiae, E. coli, and C. albicans) as revealed by MIC90 data; however there were significant variations in the MIC90 between organisms.

After 18 h of culture, MIC90 values ranged from <0.005% for C. albicans, the most susceptible organism, through to 0.31% for S. uberis and S. agalactiae, 0.625% for methicillin-resistant S. aureus, the least susceptible organisms.

After 24 h of culture, the MIC90 values were unchanged for C. albicans, S. uberis, and S. agalactiae, while the concentration of test substance required to achieve MIC90 had decreased for the other microbes tested (methicillin-sensitive S. aureus, S. pyogenes and E. coli). The concentration of test substances required to achieve MIC90 had increased to 1.25% for methicillin-resistant S. aureus.

The results indicated either persistency of antimicrobial effect or increased antimicrobial potency of the test solution over time against a broad range of organisms.

### Example 5

### Materials and Methods

A third party was contracted to perform the independent testing described herein leading to the results outlined in this specification.

### 1. Description of Test Materials and Test Methods

The methodology used in this study is based on standard MIC assay methods referenced in numerous publications.

The test formulation, comprising three novel plant extracts melaleuca oil, manuka oil, and black walnut hull extract in a specific ratio described herein, was serially diluted to give 11 different concentrations. The diluted samples were then incubated with individual microbes, and the MICs were determined as the lowest concentration of test substance that inhibited the growth of the microbe as determined by measurement of OD650 nm using a VersaMax 96 well plate reader.

### 2. Characterisation of the Test Systems:

The test systems were individualised according to the specific requirements for each of the microbes.
Tryptic soy agar (TSA) base (Bacto Difco Media Cat. No. 236940, Becton Dickinson and company)
Mueller II broth cation-adjusted (BBL media Cat. No. 212332, Becton Dickinson and company)
Defibrinated sheep blood (Cat. No CP-SHPBLD-20, Fort Richard)
Lysed horse blood (Cat. No CP LYSED 100, Fort Richard)
Staphylococcus aureus (NZCC865, ATCC12598)
Staphylococcus aureus (NZCC 4410, ATCC 33591, Strain 328 methicillin resistant)
Streptococcus pyogenes (From Dental Research Unit, Wellington Medical School)
Streptococcus uberis (NZRM 2266 NCTC 3858 (ATCC 19436, DSM 20569, NCDO 2038)
Streptococcus agalactiae (NZRM 2721, ATCC 27956 (DSM 6784))
Escherichia coli (NZRN 2220, ATCC 291214)
Candida albicans (NZRN 4356, ATCC 6450 Strain AD)
Cephalexin hydrate (Sigma Cat. No C4895)
Ciprofloxacin (Fluke Cat no. 17850)
Clarithromycin (Sigma C-9742)
Penicillin G sodium salt (Sigma P-3032)
Amphotericin B (Sigma A2942)
   3. Terminology Used with this Section
Substrate sterility controls (broth plus sample only)
Test substance free control (Mueller II broth plus inoculum)
Microbial Purity control (final microbial inoculum streaked onto agar)

### 4. Sample Preparation and Assay Conditions:

The assay procedure is tailored toward the individual requirements of the organisms.

### Preparation of Agar and Broth

Difco™ Tryptic Soy Agar (TSA) powder was added to distilled water at 40 g/L and stirred. BBL™ Mueller Hinton II Cation Adjusted broth powder was added to distilled water at 22 g/L and stirred.

TSA agar and Mueller Hinton II cation adjusted broth solutions were boiled for 1 minute with stirring to completely dissolve the powder.

TSA agar and Mueller Hinton II cation adjusted broth media were autoclaved at 121° C. for 20 minutes.

### B. Culturing on Agar

When the TSA agar solution had cooled sufficiently, 10 ml was poured into sterile 140 mm petri dishes and cooled to room temperature to allow solidification of the TSA agar gel.

The isolates of each inoculant strain was streaked from −80° C. cryostocks onto the TSA agar gel and incubated for 24-48 hrs at 37° C. until visible single colonies had developed. In the case of S. agalactiae and S. uberis, the method was modified so that when the TSA agar solution had cooled sufficiently (the bottle could be touched with the back of the hand for two seconds without discomfort), defibrinated sheep blood was added to give a 5% concentration (v/v). The blood agar was poured into a sterile 140 mm petri dish and cooled to room temperature to allow solidification of the TSA agar gel.

The Streptococcus agalactiae was streaked from −70° C. cryostocks onto the blood agar gel and incubated for 24-48 hrs at 37° C. until visible single colonies had developed.

One colony was taken with a sterile, disposable inoculating loop and used to inoculate 40 ml of Mueller Hinton II Cation Adjusted broth medium to which lysed horse blood had been added to give a 5% concentration (v/v). The inoculated broth was incubated for approximately 66 hrs at 37° C.

The broth culture was diluted with fresh, sterile Mueller Hinton II Cation Adjusted broth plus blood to an OD₆₅₀ₙₘ of approximately 0.1, equivalent to approx 10⁵ CFU/ml prior to commencement of MIC testing. This was the inoculant which was used to inoculate the test wells in each plate. The inoculant was held at 4° C. until required for plating.

The stock solutions of the test samples were prepared as described above.

Each of the two antibiotics (Cephalexin and Penicillin) was dissolved in Mueller Hinton II Cation Adjusted broth plus blood to give a final concentration of 100 μg/ml.

### C. Culturing in Broth

One colony was taken with a sterile, disposable inoculating loop from the agar and used to inoculate 40 ml of Mueller Hinton II cation adjusted broth medium. 10 ml of this seeded broth was taken and serially diluted 5 times. The inoculated broths were incubated for approximately 66 hrs at 37° C.

Following incubation, 100 ul samples of each broth culture were scanned at OD₆₅₀ₙₘ to determine a suitable broth culture which was still actively growing (in log phase).

The selected broth culture was diluted with fresh sterile Mueller Hinton II cation adjusted broth to an OD₆₅₀ₙₘ of approximately 0.1, equivalent to approx 10⁵ CFU/ml prior to commencement of MIC testing. This was the inoculant that was used to inoculate the test wells in each plate. The inoculant was held at 4° C. until required for plating.

### D. Culturing in Multi-Well Plates

Homogenised sample stock solutions of each test extract were prepared such that the concentration was 10% in Mueller Hinton II cation adjusted broth.

Each of the relevant antibiotics was dissolved in Mueller II cation adjusted broth to give a final concentration of 100 μg/ml.

The 96 well microtitre plates for each experiment were then set up as follows (See plate layouts below): To wells A1-H1 on the relevant plates 200 μl of the appropriate sample stock solution (containing 10% of sample in Mueller Hinton II cation adjusted broth or antibiotic standard) was added. To all other wells 100 μl of sterile Mueller Hinton II cation adjusted broth were added.

Using a multichannel pipette 100 μl of the extract or antibiotic was sampled from Column 1 on each plate, transferred to Column 2 and mixed thoroughly by pipetting up and down 5 times. Using fresh pipette tips 100 μl of solution was transferred from the wells of Column 2 to those of Column 3 and mixed thoroughly by pipetting up and down 5 times. At each stage the tips were discarded and the serial dilution process continued through to Column 11 on each plate. From Column 11 100 μl of solution was discarded, leaving 100 μl as the final volume in this well. This process resulted in serial double-dilutions that range from 10% to 0.01% for samples, and 100 μg/ml to 0.098 μg/ml for antibiotic standard.

100 μl of inoculant or Mueller Hinton II cation adjusted broth was added to each well as indicated on the Plate Layouts below. The addition of inoculant halved the extract concentration in each well giving final well concentrations ranging from 5.000% to 0.005% for samples and 50 μg/ml to 0.049 μg/ml for antibiotic standard.

The plates were gently tapped to ensure even mixing of the inoculant with the sample solutions.

The OD₆₅₀ₙₘ of each plate was read using a Versamax microtitre plate reader. This was recorded as the zero time (T0hours) reading.

The plates were then incubated for 16 and 24 hours at 37° C. at which point the OD₆₅₀ₙₘ of each plate was read and recorded.

Once the OD₆₅₀ₙₘ of the plates had been read, the wells containing the highest dilution of each sample (lowest concentration of test extract) without a detectable change in OD₆₅₀ₙₘ in comparison to the reading for the unsupplemented control was noted.

### Experimental Design

Each of the test samples and reference antibiotics standards were assayed in triplicate at 11 different concentrations against the bacterial isolate. On each plate, eight replicates of the unsupplemented incubation of the micro-organisms were cultured. These are in column 12 of each plate. For each test sample the first well in the plate (lowest concentration) that showed no change in OD₆₅₀ₙₘ after 16 and 24 h incubation compared with the OD₆₅₀ₙₘ of the of the inoculants only control was noted. The next highest concentration was recorded as the minimum inhibitory concentration (MIC) for the samples against the particular micro-organism. These were reported as percentage concentrations.

Table A provides proof that the ratio of compounds in preferred embodiments provides a particularly synergistic effect.

TABLE A

| | 16 h | 24 h |
|---|---|---|
| 28.8% manuka oil, 67.2% | 1.25% | 0.02% |
| melaleuca, 4% walnut | | |
| 14.4% manuka, 81.6% | 2.5% | 0.313% |
| melaleuca, 4% walnut | | |

Table B illustrates that using an extract from pine bark can also be effective as the galloyl moiety in the present invention.

TABLE B
Proof that ENZO can be interchanged for walnut as a
source of galloyl to achieve the synergistic effect

| | 16 h | 24 h |
|---|---|---|
| 28.8% manuka oil, 67.2% | 1.25% | 0.02% |
| melaleuca, 4% walnut | | |
| 28.8% manuka oil, 67.2% | 0.16% | 0.02% |
| melaleuca, 4% p. radius | | |
| pine bark extract | | |
| 28.8% manuka oil, 67.2% | 2.5% | 5% |
| melaleuca, 4% ethanol | | |

Table C illustrates a representative Composition (by volume) of varying concentrations of High Potency Melaleuca alternifolia and Leptos permum scoparium oils and a galloyl to achieve a synergistic microbial effect.

TABLE C

| Melaleuca alternifolia | Leptospermum scoparium | A galloyl source |
|---|---|---|
| 79.2 | 19.8 | 1 |
| 76 | 19 | 5 |
| 72.8 | 18.2 | 9 |
| 69.3 | 29.7 | 1 |
| 66.5 | 28.5 | 5 |
| 63.7 | 27.3 | 9 |
| 59.4 | 39.6 | 1 |
| 57 | 28 | 5 |
| 54.6 | 36.4 | 9 |

Aspects of the present invention have been described by way of example only and it should be appreciated that modifications and additions may be made thereto without departing from the scope of the appended claims.

What we claim is:

1. An antimicrobial composition, comprising:
a) melaleuca oil, or an isolated fraction or an active agent or equivalent thereof;
b) manuka oil, or an isolated fraction or an active agent or equivalent thereof; and
c) at least one galloyl moiety.

2. An antimicrobial composition as claimed in claim 1, wherein the galloyl moiety is present in walnut hull, or an isolated fraction or an active agent or equivalent thereof.

3. An antimicrobial composition as claimed in claim 2 wherein the walnut hull is black walnut hull.

4. An antimicrobial composition as claimed in claim 1 wherein the galloyl moiety is derived from pine bark extract, or an isolated fraction or an active agent or equivalent thereof.

5. An antimicrobial composition as claimed in claim 4 wherein the pine is Pinus radiata.

6. An antimicrobial composition as claimed in claim 1 wherein the melaleuca oil is present in the order of 62% to 72% v/v.

7. An antimicrobial composition as claimed in claim 6 when the melaleuca oil is present in the order of 67% v/v.

8. An antimicrobial composition as claimed in claim 1 wherein the manuka oil is present in the range of 24% to 34% v/v.

9. An antimicrobial composition as claimed in claim 8 wherein the manuka oil is present in the order of 29% v/v.

10. An antimicrobial composition as claimed in claim 1 wherein the galloyl moiety is present in the order of 2% to 6% v/v.

11. An antimicrobial composition as claimed in claim 10 wherein the galloyl moiety is present in the order of 4% v/v.

12. A method of manufacturing an antimicrobial composition, comprising:
combining:
a) melaleuca oil, or an isolated fraction or an active agent thereof;
b) manuka oil, or an isolated fraction or an active agent thereof; and
c) at least one galloyl moiety.

13. A method of manufacturing an antimicrobial composition as claimed in claim 12, wherein the galloyl moiety is presented in walnut hull, or an isolated fraction or an active agent or equivalent thereof.

14. A method of manufacturing an antimicrobial composition as claimed in claim 13 wherein the walnut hull is black walnut hull.

15. A method of manufacturing an antimicrobial composition as claimed in claim 12 wherein the galloyl moiety is derived from pine bark extract, or an isolated fraction or an active agent or equivalent thereof.

16. A method of manufacturing an antimicrobial composition as claimed in claim 15 wherein the pine is Pinus radiata.

17. A method of manufacturing an antimicrobial composition as claimed in claim 12 wherein the melaleuca oil is present in the order of 62% to 72% v/v.

18. A method of manufacturing an antimicrobial composition as claimed in claim 17 when the melaleuca oil is present in the order of 67% v/v.

19. A method of manufacturing an antimicrobial composition as claimed in claim 12 wherein the manuka oil is present in the range of 24% to 34% v/v.

20. A method of manufacturing an antimicrobial composition as claimed in claim 19 wherein the manuka oil is present in the order of 29% v/v.

21. A method of manufacturing an antimicrobial composition as claimed in claim 12 wherein the galloyl moiety is present in the order of 2% to 6% v/v.

22. A method of manufacturing an antimicrobial composition as claimed in claim 21 wherein the galloyl moiety is present in the order of 4% v/v.

23. A use in the manufacture of the anti-microbial composition as claimed in claim 1 for the prevention or treatment of a microbial infection in an animal in need thereof.

24. A use in the manufacture of any of the anti-microbial compositions as claimed in claim 23 wherein the microbial infection is Pseudomonas aeruginosa.

25. A use in the manufacture of any of the anti-microbial compositions as claimed in claim 23 wherein the microbial infection is Staphylococcus. aureus.

26. A method of preventing or treating a microbial infection in an animal, comprising the step of:
administering to the animal a therapeutically effective amount of the antimicrobial composition of claim 1.

27. A method as claimed in claim 26 when the microbial infection is Pseudomonas aeruginosa.

28. A method of preventing or treating a microbial infection in an animal as claimed in claim 26 wherein the infection is Staphylococcus. aureus.
